(19) United States
(12) Patent Application Publication
(10) Pub. No.: US 2026/0001061 A1
(43) Pub. Date: Jan. 1, 2026

(54) THERMOCATALYSIS METHOD FOR PREPARING GLYCOLIC ACID FROM WASTE PLASTICS BY ONE-POT, AND CATALYST

(71) Applicant: CHINA UNIVERSITY OF PETROLEUM (EAST CHINA), Qingdao (CN)

(72) Inventors: Hao YAN, Qingdao (CN); Qinghai CHEN, Qingdao (CN); Xiang FENG, Qingdao (CN); Kai ZHAO, Qingdao (CN); Yibin LIU, Qingdao (CN); Xin ZHOU, Qingdao (CN); De CHEN, Qingdao (CN); Chaohe YANG, Qingdao (CN)

(73) Assignee: CHINA UNIVERSITY OF PETROLEUM (EAST CHINA), Qingdao (CN)

(21) Appl. No.: 19/251,990

(22) Filed: Jun. 27, 2025

(30) Foreign Application Priority Data
Jun. 28, 2024 (CN) 202410864788.4

(51) Int. Cl. B01J 23/66; B01J 35/45; B01J 37/00; B01J 37/04; B01J 37/08; C07C 51/09

(52) U.S. Cl. CPC B01J 23/66; B01J 35/45; B01J 37/009; B01J 37/04; B01J 37/08; C07C 51/09

(57) ABSTRACT

A method for preparing glycolic acid from waste polyester plastic comprises: (1) mixing waste polyester plastic, a hydrolyzing agent, a solvent, and a catalyst in a reactor, and then adding a gas containing oxygen for reaction; (2) filtering and washing a reaction product to obtain terephthalic acid and oxidation product, wherein the oxidation product comprises glycolic acid. The catalyst comprises a metal element Au and a second metal oxide, and the second metal oxide is one compound or more compounds selected from a group consisting of NiO, TiO₂, CeO₂, ZrO₂, and MgO. The catalyst can be used for depolymerizing and oxidizing waste polyester plastics in one pot to prepare glycolic acid.

### REFERENCE TO RELATED APPLICATIONS

The application claim the priority right of Chinese Patent Application No. 202410864788.4 filed with the State Intellectual Property Office of China on Jun. 28, 2024, the entire content of which is incorporated herein by reference.

### TECHNICAL FIELD

This application relates to a catalytic method for PET hydrolysis and selective oxidation of EG, and specifically to a catalytic method for hydrolyzing PET to obtain a solid-phase product TPA and selectively catalytically oxidizing a liquid-phase product ethylene glycol to glycolic acid.

### BACKGROUND ART

Polyethylene terephthalate (PET) is the polyester material with the largest global consumption, and has properties of high rigidity, high transparency and good heat resistance. It is widely used in packaging, textiles, chemical industry and other fields. The problem of waste PET disposal is becoming increasingly prominent due to the rapid increase in global PET production and consumption. A large amount of accumulated PET waste causes serious pollution and damage to the environment. It is difficult to deal with the increasing amount of PET waste by the existing methods for recycling PET, so it is crucial to exploit new and greener recycling routes to generate high value-added products.

Glycolic acid (GA) is the simplest a-hydroxy acid. In the fields of cosmetics, detergents and medicine, the products relative to GA have excellent properties such as biodegradability, biocompatibility, and low toxicity, so the market demand of the products is significant high. At present, the most common process for industrially preparing glycolic acid is formyl carbonylation reaction, however there are the disadvantages of requiring high temperature and high pressure in the reaction, and being environmentally unfriendly. Toxic and explosive organic solvents such as acetone and tri-n-octylamine are used for purifying the product in the process, so it is increased in industrial costs and caused to pollute the environment, and safety hazards can be happened. Therefore, there is a need to develop a low-cost, green, environmentally friendly, and large-scale method for producing glycolic acid.

The method for converting waste polyethylene glycol esters such as PET into glycolic acid can not only effectively reduce environmental pollution and save fossil energy, but also bring huge economic benefits. It has been achieved to converting waste PET to glycolic acid by electrocatalysis and enzymatic catalysis. For example, Chinese patent application publication No. CN114008211A discloses a method for producing high value-added compounds from polyethylene terephthalate including: producing terephthalic acid and ethylene glycol by hydrolysis of polyethylene terephthalate; and converting terephthalic acid into one or more compounds selected from the group consisting of gallic acid, pyrogallol, catechol, muconic acid, and vanillic acid by the action of a biocatalyst, wherein protocatechuic acid is an intermediate produced by biotransformation, or glycolic acid is prepared by fermentation of ethylene glycol.

In the method for preparing glycolic acid from PET in the prior art, PET is first depolymerized into monomers, and then ethylene glycol obtained by the depolymerizing process is converted into glycolic acid in an electrolytic cell or under the action of microorganisms. In the two-step method, there are the advantages of lower production efficiency and higher cost of electrocatalysis and enzymatic catalysis, so it is limited in the large-scale industrial application.

### SUMMARY OF THE INVENTION

In one aspect, the present application is to provide an Au-based catalyst. Waste polyester plastic is depolymerized and oxidized by one-pot method to prepare glycolic acid under the thermocatalytic action of the Au-based catalyst.

In a second aspect of the present application, the catalytic performance on oxidation of ethylene glycol to glycolic acid is improved in the alkaline hydrolysis conditions of waste polyester plastic through the action of Au-based catalyst, and the yield of glycolic acid is high in the mild condition.

In a third aspect of the present application, waste polyester plastic as raw materials is catalytically oxidized to glycolic acid in one step. So waste PET plastics can be degraded and reused, and glycolic acid with high-value is obtained.

A catalyst for preparing glycolic acid from waste polyester plastic, comprises: a metal element Au and a second metal oxide, wherein the second metal oxide is one compound or more compounds selected from a group consisting of NiO, TiO₂, CeO₂, ZrO₂, and MgO.

The catalyst has good catalytic performance and good stability in a strongly alkaline environment, and still maintains good catalytic performance after long-term use.

A method for preparing glycolic acid by the above catalyst comprises: mixing waste polyester plastic, a solvent, and the above catalyst in a kettle-type reactor, and then adding a gas containing oxygen for reacting to obtain glycolic acid.

In the application, the hydrolysis of waste polyester plastic and the catalytic oxidation of ethylene glycol to glycolic acid are carried out in the same reaction system, so there is no need of separating the hydrolysis products of waste polyester plastic, and the selectivity of glycolic acid is high. The process flow is greatly simplified. On the other hand, waste plastics can be recycled, protecting the ecological environment and reducing the cost of producing glycolic acid.

The above catalyst can also be directly used in the reaction system for preparing glycolic acid from ethylene glycol as a raw material. Under the thermal catalytic action of Au-based catalyst, ethylene glycol can be oxidized to prepare glycolic acid at relatively low reaction temperatures.

### BRIEF DESCRIPTION OF THE DRAWINGS

FIG. 1a is the transmission electron micrograph of Au-based catalyst prepared in embodiment 1.

FIG. 1b is the transmission electron micrograph of Au-based catalyst prepared in embodiment 2.

FIG. 1c is the transmission electron micrograph of Au-based catalyst

prepared in embodiment 3.

FIG. 1d is the transmission electron micrograph of Au-based catalyst prepared in embodiment 4.

FIG. 1f is the transmission electron micrograph of Au-based catalyst prepared in embodiment 5.

FIG. 2 is an Au-XPS spectrum of Au-based catalysts prepared in embodiment 1 to 5.

FIG. 3 is a HPLC spectrum of the product glycolic acid prepared by Au/NiO with loading 0.5%.

### DETAILED DESCRIPTION

The Au-based catalyst of the present application and the application thereof are further described in detail below. The protection scope of the present application is not limited, and the protection scope is defined by the claims. The disclosed details are used for thoroughly understanding the various disclosed embodiments. However, one skilled in the art can know that embodiments may be implemented without one or more of these details, and with other materials and the like.

Unless the context requires, in the specification and claims, the term “comprising” or “containing” should be interpreted as an open-end and inclusive meaning, that is, “including, but not limited to”.

The “embodiment”, “an embodiment”, “another embodiment” or “some embodiments” mentioned in the specification refers to that the specific features, structures or characteristics described in the embodiment are included in at least one embodiment. Thus, “an embodiment,” “an embodiment,” “another embodiment,” or “some embodiments” are not necessarily all included in the same embodiment. Furthermore, the specific features, structures or characteristics may be combined in any manner in one or more embodiments. Each feature disclosed in the specification may be replaced by any alternative feature that can have the same, equivalent or similar function. Therefore, unless otherwise specified, the disclosed features are only general examples of equivalent or similar features.

The present application is further described below in conjunction with specific Embodiments. It should be understood that these Embodiments are used for illustrating only the solutions, and are not intended to limit the scope of the present application. Experimental methods not being specified with specific conditions in the following Embodiments are usually in accordance with conventional conditions or conditions recommended by the manufacturers. Unless otherwise stated, all percentages, ratios, proportions, or parts are by weight.

The waste polyester plastic of the application includes but is not limited to polyethylene terephthalate (PET), polyethylene furanoate (PFE), polyethylene naphthalate (PEN), or polyethylene adipate (PEA). For example, PET can be polyethylene terephthalate material with any number average molecular weight, and more specifically, waste polyester plastic of the application is polyethylene terephthalate (PET) with a weight average molecular weight in a range of 16000 g/mol to 30000 g/mol.

EG is an abbreviation for ethylene glycol, and GA is an abbreviation for glycolic acid.

PVA is an abbreviation for polyvinyl alcohol.

The “loading” of Au refers to the mass percentage of Au element to the second metal oxide.

The unit of weight/volume percentage in the present application is well known to those skilled in the art, for example, it refers to the weight of solute in 100 milliliters of solution.

In the present application, the concentration unit “M” of the solution represents mol/L.

Unless defined otherwise, all technical terms used herein have the same meaning as commonly understood by those skilled in the art. In addition, any methods and materials similar or equivalent to those recited can be applied in the method of the present application. The preferred implementation methods and materials described herein are only used for illustrating the application.

The catalyst of the application can be used for catalytic oxidation of EG to GA, or used for alkaline hydrolysis depolymerization of waste polyester plastic in tandem with efficient oxidation of EG to prepare GA, so the product with high value is obtained, and the hydrolysis reaction is further improved in the chemical equilibrium of waste polyester plastic. The waste polyester plastic is rapidly depolymerized in a short time (<3 h). The process has good applicability and economy in applying in industrial production.

In the existing system for preparing glycolic acid by catalytic oxidation of ethylene glycol, the reaction environment is relatively mild, and the requirements for the performance of the catalyst are relatively low. However when applied to the alkaline hydrolysis system of PET, the performance of the catalyst is poor or easily to be deactivated.

In view that the catalyst for liquid-phase catalytic oxidation of ethylene glycol (EG) has poorly catalytic oxidation performance on the traditional hydrolysis depolymerization of waste polyester plastic in the harsh reaction conditions, a catalyst is provided to improve the selective oxidation of EG obtained by be depolymerized in the liquid phase under the alkaline hydrolysis depolymerization conditions of waste polyester plastic. A method for preparing a catalyst used for catalytic oxidation of EG in the liquid phase under the harsh reaction conditions of waste polyester plastic hydrolysis depolymerization is provided, so waste polyester plastic hydrolysis depolymerization and efficient selective oxidation of EG are carried out in the same system to generate glycolic acid.

A catalyst for preparing glycolic acid from waste polyester plastic, comprises: a metal element Au and a second metal oxide, wherein the second metal oxide is one compound or more compounds selected from a group consisting of NiO, TiO₂, CeO₂, ZrO₂, and MgO.

In the catalyst, a loading of Au is in a range of 0.5 wt % to 10.0 wt %.

In some embodiment, the loading of Au is in a range of 0.5 wt % to 5.0 wt %.

Under the synergism of the metal Au and the second metal oxide, the stability of the catalyst is good under the alkaline condition. The catalyst exhibits good metal dispersion and catalytic performance in the oxidation reaction of EG in the alkaline hydrolysis system of waste polyester plastic. Due to the synergism of two metals, the catalyst formed by two metals has better catalytic performance.

In some embodiment, the second metal oxide is one compound or more compounds selected from a group consisting of NiO, TiO₂, and MgO.

Optionally, the second metal oxide is one compound or more compounds selected from a group consisting of NiO and TiO₂.

In some embodiment, a size of Au particles of the catalyst for preparing glycolic acid from waste polyester plastic is smaller than 10 nm. Optionally, the size of Au particles of the catalyst is smaller than 5 nm.

Especially under the synergism of Au and Ni oxide in the catalyst, the catalytic activity for ethylene glycol oxidation to glycolic acid is enhanced. At a lower temperature, PET can be converted into glycolic acid under the action of the catalyst, and the selectivity of glycolic acid is high.

The catalyst for preparing glycolic acid from waste polyester plastic of the application can be prepared by any method in the prior art.

In the application, optionally, a method for preparing a catalyst for preparing glycolic acid from waste polyester plastic comprises: mixing gold sol and the second metal oxide to obtain a first mixture, filtering the first mixture to obtain a solid substance, and drying and calcining the solid substance to obtain the catalyst.

A calcination temperature is in a range of 200° C. to 300° C.

When the calcination temperature is too high, Au particles are be aggregated to cause an increase in the size of Au particles, and the increase in the size of Au particles causes a decrease in catalytic performance.

A method for preparing the gold sol comprises: mixing a water-soluble compound containing Au element and a dispersant in water to obtain a second mixture, and mixing the second mixture with a reducing agent to obtain the gold sol.

The reducing agent comprises but is not limited to sodium borohydride or ascorbic acid, etc.

The dispersant comprises PVA or/and polyvinylpyrrolidone. Other substances can also be selected as the dispersant for dispersing the Au element well.

Generally, a mass of the dispersant is in a range of 0.5 to 4 times a mass of the Au element.

In some embodiment, the reducing agent can be sodium borohydride.

A molar ratio of the reducing agent to Au element is greater than or equal to 3:1. Optionally, the molar ratio of the reducing agent to Au element is greater than or equal to 4:1.

In the process of preparing the gold sol, the dispersant and water are first mixed at a temperature of 60° C.-80° C, and it is cooled to room temperature after the dispersant is dissolved.

The room temperature refers to a temperature of 15° C. to 30° C.

The water-soluble compound containing Au element comprises chloroauric acid.

In another aspect, a method for preparing glycolic acid of the present application comprises:
(1) mixing waste polyester plastic, a hydrolyzing agent, a solvent, and a catalyst in a kettle-type reactor, and then adding a gas containing oxygen for reaction;
(2) filtering and washing a reaction product to obtain terephthalic acid and oxidation product, wherein the oxidation product comprises glycolic acid.

In some embodiment, in step (1), a reaction temperature is in a range of 100° C. to 170° C.

Optionally, in step (1), the reaction temperature is in a range of 100° C. to 150° C.

As an alternative, in step (1), the reaction temperature is in a range of 120° C. to 140° C.

In step (1), a reaction time is equal to or more than 1 h.

As an optional solution, in step (1), the reaction time is in a range of 1 h to 6 h.

As an optional solution, in step (1), the reaction time is in a range of 2.5 h to 6 h.

The hydrolyzing agent is one compound or more compounds selected from a group consisting of KOH, Ca(OH)₂, CaCO₃, and NaOH; preferably, the hydrolyzing agent is NaOH or KOH.

A weight ratio of the hydrolyzing agent to the waste polyester plastic is in a

range of 0.5:1 to 1:1. Optionally a weight ratio of NaOH to the waste polyester plastic is 0.8:1.

The gas containing oxygen comprises O2 or air.

In the method for preparing glycolic acid of the present application, the strong alkaline hydrolyzing agents NaOH and KOH can provide sufficient OH⁻ in the reaction solution to break the ester bonds of the waste polyester plastic. The selective oxidation of EG is inhibited by the excessive alkali. In different atmospheres, the waste polyester plastic is completely depolymerized to obtain high yields of dicarboxylic acid monomers. By analyzing the liquid phase components, it is shown that EG is effectively converted into glycolic acid at 3 Mpa by adding O₂ to the reactor. The metal oxide of the catalyst of the present application has a better activation effect on oxygen than multi-walled carbon nanotubes (CNF). Ethylene glycol reacts with oxygen at the interface between Au and the metal oxide, and ethylene glycol is accelerated to be converted into glycolic acid under the synergism and electronic interaction of Au and the metal oxide. In particular, for Au/NiO catalyst, it is more conducive to the adsorption of EG and the formation of intermediates during the reaction, and the yield of glycolic acid are higher.

In some embodiment, in the method, the catalyst accounts for 5% to 10% of the mass of the waste polyester plastic.

In some embodiment, in the method, a weight ratio of the waste polyester plastic to water is in a range of 1:5 to 1:20.

The waste polyester plastic can be PET. Optionally, PET is polyethylene terephthalate with a weight-average molecular weight of 16000 to 3000.

After the reaction is completed, it is subject to filtration and washing to obtain a solid phase product and a liquid phase product, and the liquid phase product is subject to distillation separation.

In the present application, the waste polyester plastic as raw materials is hydrolyzed in alkaline condition and subject to catalytic oxidation of ethylene glycol simultaneously in the same reactor to produce glycolic acid. In a relatively mild temperature (100° C. to 170° C.) and strong alkaline reaction system, the conversion rate of ethylene glycol and the selectivity of glycolic acid are very high, and the thermal stability is good. Moreover, the hydrolysis rate of waste polyester plastic per unit time is fast, and the amount of glycolic acid is large.

The following further illustrates the solutions of the present application in conjunction with specific embodiments.

Nickel oxide, titanium dioxide, magnesium oxide, cerium oxide, and zirconium oxide in embodiments were all purchased from Sinopharm Group.

### Embodiment 1

0.01 g of PVA (alcoholysis degree of 98%, and degree of polymerization of Polyvinyl alcohol: 1750+50) was added in 200 ml of deionized water, heated up to 80° C. and stirred for 2 h, and cooled to room temperature. 0.5 ml of chloroauric acid solution with a concentration of 0.02 g/ml was mixed with PVA solution and stirred for 30 min, and then 3 ml of sodium borohydride solution with a concentration of 0.1 mol/L was added in the mixed solution to obtain a gold sol. 1 g of commercial nickel oxide was added in the gold sol and stirred for 10 h to obtain a mixture, the mixture was filtered to get filtration cake, and the filtration cake was dried at 80° C. overnight and calcined at 250° C. for 2 h to obtain Au/NiO catalyst. The loading of Au in the obtained catalyst was 0.5 wt %.

### Embodiment 2

The preparation of the gold sol was the same as in Embodiment 1. 1 g of commercial titanium dioxide was added in the gold sol and stirred for 10 h to obtain a mixture, the mixture was filtered to get filtration cake, and the filtration cake was dried at 80° C. overnight and calcined at 250° C. for 2 h to obtain Au/TiO₂ catalyst. The loading of Au in the obtained catalyst is 0.5 wt %.

### Embodiment 3

The preparation of the gold sol was the same as in Embodiment 1. 1 g of commercial magnesium oxide was added in the gold sol and stirred for 10 h to obtain a mixture, the mixture was filtered to get filtration cake, and the filtration cake was dried at 80° C. overnight and calcined at 250° C. for 2 h to obtain Au/MgO catalyst. The loading of Au in the obtained catalyst is 0.5 wt %.

### Embodiment 4

The preparation of the gold sol was the same as in Embodiment 1. 1 g of commercial cerium oxide was added in the gold sol and stirred for 10 h to obtain a mixture, the mixture was filtered to get filtration cake, and the filtration cake was dried at 80° C. overnight and calcined at 250° C. for 2 h to obtain Au/CeO₂ catalyst. The loading of Au in the obtained catalyst is 0.5 wt %.

### Embodiment 5

The preparation of the gold sol was the same as in Embodiment 1. 1 g of commercial zirconium oxide was added in the gold sol and stirred for 10 h to obtain a mixture, the mixture was filtered to get filtration cake, and the filtration cake was dried at 80° C. overnight and calcined at 250° C. for 2 h to obtain Au/ZrO2 catalyst. The loading of Au in the obtained catalyst is 0.5 wt %.

The waste plastics used in embodiments 6 to 8 and comparative examples 1 to 3 were all PET with a weight-average molecular weight of approximately 30,000 g/mol.

### Embodiment 6

In the embodiment, the catalysts prepared in embodiments 1 to 5 were used for preparing glycolic acid from PET with a weight-average molecular weight of approximately 30,000 g/mol. The method for preparing glycolic acid comprises:

uniformly mixing 1 g of PET waste plastic, 20 ml of deionized water, 0.8 g of sodium hydroxide, and 0.1 g of catalyst to obtain a mixture, placing the mixture in a 50 ml high-pressure reactor, and adding oxygen into the reactor to the pressure inside the reactor with 1 Mpa, and carrying out reaction at 130° C. for 3 h with fully stirring; after the reaction was completed, cooling the reactor to room temperature, filtering the mixed solution in the reactor to obtain a first filter cake and a first filtrate; adding 0.5 mol/L hydrochloric acid solution in the first filtrate to adjust the solution to pH=3 to precipitate terephthalic acid; filtering terephthalic acid out to obtain a filter cake of terephthalic acid and a second filtrate; distilling the second filtrate under reduced pressure to obtain glycolic acid. The reaction results were shown in Table 1.

The product glycolic acid was qualitatively and quantitative analyzed by means of a High performance liquid chromatography (Shimadzu LC-20AT) equipped with a UV detector (SPD-M20A) and a differential refractive index detector (RID-20A). The conditions were as the follows: a mobile phase of 0.005 mol/L sulfuric acid solution, a flow rate of 0.17 mL/min, a chromatography column model of Rezex ROA organic acid H+(8%), 300×7.8 mm, and a detecting temperature of 35° C. For example, the formation of the product glycolic acid prepared by Au/NiO with loading 0.5% was shown in FIG. 3.

### Embodiment 7

In the embodiment, the catalyst prepared in embodiment 1 is used for preparing glycolic acid from PET with a weight-average molecular weight of approximately 30,000 g/mol. The process steps and process conditions refer to embodiment 6, except that the reaction time was 4 h. The reaction results were shown in Table 1.

### Embodiment 8

In the embodiment, the catalyst prepared in embodiment 1 is used for preparing glycolic acid from PET with a weight-average molecular weight of approximately 30,000 g/mol. The process steps and process conditions refer to embodiment 6, except that the reaction time was 1 h. The reaction results were shown in Table 1.

### Comparative Example 1

The method for preparing glycolic acid from waste plastic PET in comparative example 1 refers to embodiment 6, except that no catalyst was added. The reaction results were shown in Table 1.

### Comparative Example 2

The method for preparing glycolic acid from waste plastic PET in the comparative example 2 refers to embodiment 6, except that the catalyst was changed to 0.1 g AuCu/CNT (the method for preparing the catalyst AuCu/CNT refers to the content recited in Catalytic upgrading of PET to acid by engineering bimetallic AuCu/CNT catalyst; DOI: 10.1002/jctb.7489, wherein the loading of gold was 2.5 wt % and the loading of copper was 0.5 wt %). The reaction results were shown in Table 1.

### Comparative Example 3

The method for preparing glycolic acid from waste plastic PET in the comparative example 3 refers to embodiment 6, except that no catalyst was added and the reaction time was adjusted to 1h. The reaction results were shown in Table 1.

| | | | Tere- | | |
|---|---|---|---|---|---|
| | | Conversion | phthalic | EG | GA |
| Embodi- | Catalyst | rate of | acid Yield | Yield | Yield |
| ment | Sample | plastic (%) | (%) | (%) | (%) |
| Embodi- | Au/NiO with | 100% | 99.8% | 12.7% | 78.2% |
| ment 6 | loading 0.5% | | | | |
| | Au/TiO₂ with | 100% | 99.3% | 16.8% | 70.7% |
| | loading 0.5% | | | | |
| | Au/MgO with | 100% | 99.5% | 30.1% | 55.3% |
| | loading 0.5% | | | | |
| | Au/CeO₂ with | 100% | 99.3% | 45.9% | 43.6% |
| | loading 0.5% | | | | |
| | Au/ZrO₂ with | 100% | 99.7% | 50.3% | 37.5% |
| | loading 0.5% | | | | |
| Embodi- | Au/NiO | 100% | 99.8% | 0.1% | 87.5% |
| ment 7 | loading with | | | | |
| | 0.5% | | | | |
| Embodi- | Au/NiO | 61.4% | 56.4% | 18.1% | 35.1% |
| ment 8 | loading with | | | | |
| | 0.5% | | | | |
| Compar- | None | 100% | 99.2% | 98.3% | 0.2% |
| ative | | | | | |
| Example 1 | | | | | |
| Compar- | AuCu/CNT | 100% | 99.5% | 75.6% | 18.6% |
| ative | | | | | |
| Example 2 | | | | | |
| Compar- | None | 50.2% | 47.6% | 42.3% | 0.2% |
| ative | | | | | |
| Example 3 | | | | | |

$$Conversion rate of plastic ( \% ) = {C}_{PET} \% = \frac{{m}_{0} + {m}_{cat} - {m}_{1}}{{m}_{0}} \times 100 \%$$

m_{cat} refers to the mass of catalyst added (determined to remain constant before and after the catalyst reaction), measured in grams; m₀ refers to the initial mass of PET added, measured in grams; m₁ is the mass of the first filter cake after drying.

$$Terephthalic acid Yield ( \% ) = TPA \% = \frac{m \times 166.13}{\frac{{m}_{0}}{192}} \times 100 \%$$

In the formula, m refers to the mass of the filter cake of terephthalic acid after drying, in grams; m₀ refers to the initial added mass of PET, in grams; 166.13 is the relative molecular weight of terephthalic acid; 192 is the relative molecular weight of a chain link in PET.

$$EG Yield ( \% ) = EG yield ( \% ) = \frac{{N}_{EG \cdot yield}}{\frac{{m}_{0}}{192}} \times 100 \%$$

$$GA Yield ( \% ) = GA yield ( \% ) = \frac{{N}_{GA \cdot yield}}{\frac{{m}_{0}}{192}} \times 100 \%$$

$${N}_{GA . yield} , {N}_{EG . yield} ,$$

N_{OA.yield} respectively represent the molar amounts of glycolic acid and ethylene glycol in the product; m₀ refers to the initial added mass of PET.

### Experimental Example 1

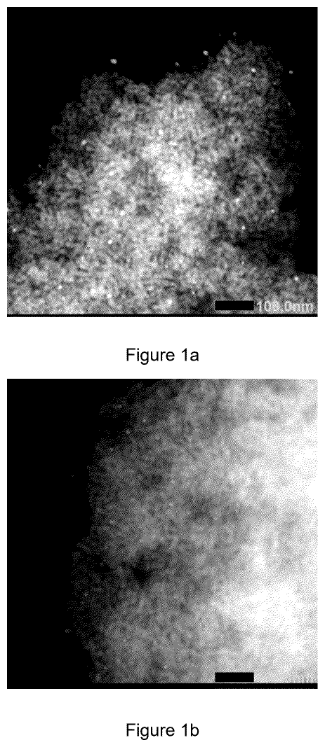

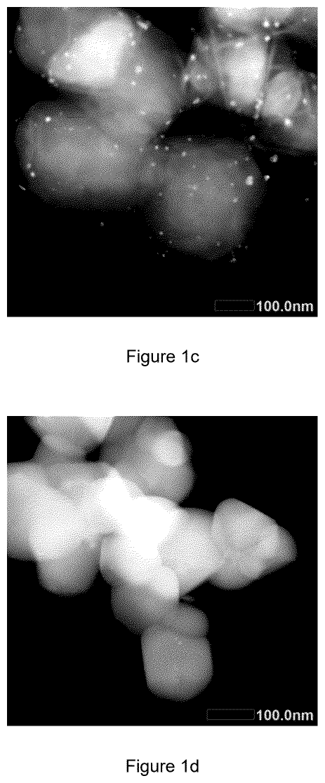

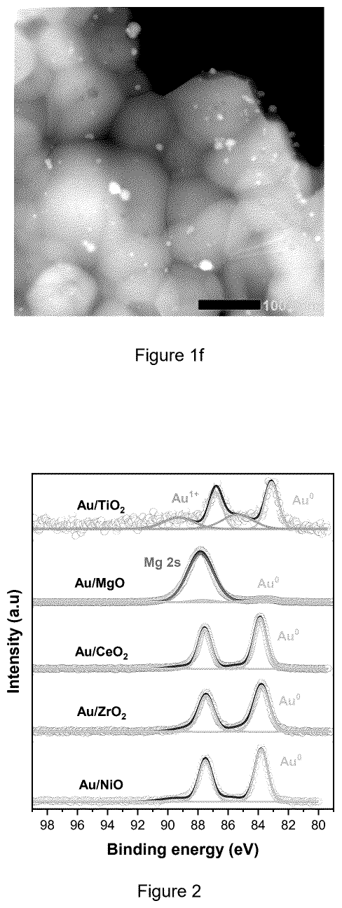

Transmission electron microscopy (TEM) analysis was performed on the catalysts prepared in embodiments 1 to 5, as shown in FIGS. 1a to 1f. FIG. 1a was the TEM of Au/NiO catalyst of embodiment 1, FIG. 1b was the TEM of Au/TiO₂ catalyst of embodiment 2, FIG. 1c was the TEM of Au/MgO catalyst of embodiment 3, FIG. 1d was the TEM of Au/CeO₂ catalyst of embodiment 4, and FIG. 1f was the TEM of Au/ZrO₂ catalyst of embodiment 5. It can be observed that Au was uniformly dispersed on the surface of the catalyst, and the particle size of Au species on the catalyst was similar (˜5 nm).

Under the condition that the Au particle size is approximately the same, the Au-based catalysts exhibit different catalytic efficiencies, which indicate that the support plays an important role in the reaction.

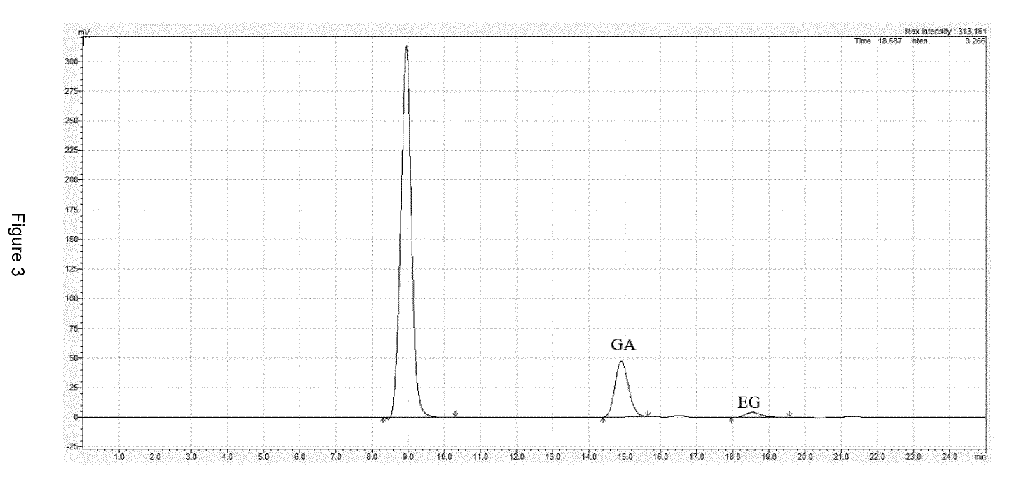

X-ray photoelectron spectroscopy (XPS) analysis was performed on the catalysts prepared in embodiments 1 to 5. The Au-2P orbital XPS spectra were shown in FIG. 2. The Au 4f 7/2 was around 84.0 eV. Au was mainly in the form of an elementary substance, and the Au binding energy on the catalyst shifted to the right. Compared with the standard Au elementary substance, the Au binding energy on the catalyst was decreased, which indicates that Au exhibits an electron-rich state, and the support transfers electrons to the Au particles.

What is claimed is:

1. A method for preparing glycolic acid from waste polyester plastic, comprising:
(1) mixing waste polyester plastic, a hydrolyzing agent, a solvent, and a catalyst in a reactor, and then adding a gas containing oxygen for reaction;
(2) filtering and washing a reaction product to obtain terephthalic acid and oxidation products;
wherein the oxidation products comprises glycolic acid; and
wherein the catalyst comprises a metal element Au and a second metal oxide, and the second metal oxide is one compound or more compounds selected from a group consisting of NiO, TiO₂, CeO₂, ZrO₂, and MgO.

2. The method according to claim 1, wherein a loading of Au is in a range of 0.5 wt % to 10.0 wt %.

3. The method according to claim 1, wherein the second metal oxide is one compound or more compounds selected from a group consisting of NiO, TiO₂, and MgO.

4. The method according to claim 1, wherein a size of Au particles of the catalyst for preparing glycolic acid from waste polyester plastic is smaller than or equal to 10 nm.

5. The method according to claim 1, wherein a size of Au particles of the catalyst for preparing glycolic acid from waste polyester plastic is smaller than or equal to 5 nm.

6. The method according to claim 1 wherein the second metal oxide is one compound or more compounds selected from a group consisting of NiO and TiO₂.

7. The method according to claim 1, wherein in step (1), a reaction temperature is in a range of 100° C. to 170° C.

8. The method according to claim 1, wherein in step (1), a reaction temperature is in a range of 120° C. to 140° C.

9. The method according to claim 7, wherein in step (1), a reaction time is equal to or more than 1 h.

10. The method according to claim 7, wherein in step (1), a reaction time is in a range of 2.5 h to 6 h.

11. The method according to claim 1, wherein the hydrolyzing agent is one compound or more compounds selected from a group consisting of KOH, Ca(OH)₂, CaCO₃, and NaOH.

12. The method according to claim 1, wherein a weight ratio of the waste polyester plastic to water is in a range of 1:5 to 1:20.

13. The method according to claim 1, wherein the gas containing oxygen is O₂, and a pressure of the reactor is in a range of 1 MPa to 3 MPa.

14. The method according to claim 1, wherein the catalyst accounts for 5% to 10% of a mass of the waste polyester plastic.

15. The method according to claim 1, wherein a method for preparing the catalyst comprises:
mixing gold sol and the second metal oxide to obtain a first mixture;
filtering the first mixture to obtain a solid substance; and
drying and calcining the solid substance to obtain the catalyst.

16. The method according to claim 15, wherein the calcination temperature is in a range of 200° C. to 300° C.

17. The method according to claim 15, wherein a method for preparing the gold sol comprises:
mixing a water-soluble compound containing Au element and a dispersant in water to obtain a second mixture;
mixing the second mixture with a reducing agent to obtain the gold sol; and
wherein a molar ratio of the reducing agent to Au element is greater than or equal to 3:1.

18. The method according to claim 15, wherein the dispersant is selected from PVA or polyvinylpyrrolidone; and wherein a mass of the dispersant is in a range of 0.5 to 4 times a mass of the Au element.

19. The method according to claim 1, wherein the waste polyester plastic comprises polyethylene terephthalate, polyethylene furanoate, polyethylene naphthalate, or polyethylene adipate.
